(19) United States
(12) Patent Application Publication
(10) Pub. No.: US 2023/0293414 A1
(43) Pub. Date: Sep. 21, 2023

(54) USE OF CYCLIN-DEPENDENT KINASE (CDK) INHIBITORS FOR THE SLOWDOWN OR PREVENTION OF BIOLOGICAL AGING

(71) Applicant: HOPE PERMANENTE, LLC, Santa Fe, NM (US)

(72) Inventor: Felipe Veloso, Santiago (CL)

(21) Appl. No.: 18/040,637

(22) PCT Filed: Aug. 2, 2021
(86) PCT No.: PCT/US2021/044153

§ 371 Date: Feb. 3, 2023

Related U.S. Application Data
(60) Provisional application No. 63/062,705, filed on Aug. 7, 2020.

(51) Int. Cl. A61K 8/49; A61Q 19/08

(52) U.S. Cl. CPC A61K 8/4953; A61Q 19/08

(57) ABSTRACT

The present disclosure provides the use of cyclin-dependent kinase (CDK) inhibitors, specifically CDK9 and/or CDK5 inhibitors, in the manufacture of compositions for the slowdown or prevention of biological aging. The inhibition of CDK9 and/or CDK5 activity in particular prevents the phosphorylation of DNA-proximal amino acid residues in the linker histone variants H1.0 (also known as H1 histone family, member 0) and H1x (also known as H1 histone family, member X). Because these specific phosphorylation events are prevented, critical higher-order constraints on chromatin dynamics in adult cells are significantly stabilized-- a time-related imbalance of these constraints has been proposed to be the fundamental cause of biological aging. The resulting stabilization of higher-order constraints on chromatin dynamics thus makes the use of CDK9 and/or CDK5 inhibitors suitable for conferring significant resistance to biological aging.

### TECHNICAL FIELD OF THE INVENTION

The present invention relates to the use of specific cyclin-dependent kinase (CDK) inhibitors to slow down or prevent biological aging, which in turn encompasses clinical and/or cosmetic applications.

### BACKGROUND OF THE INVENTION

From services of cryonic suspension and storage of humans and pets to “anti-aging” skin care cosmetics, a variety of industries try to satisfy the increasing human demand to slow down or even prevent biological aging. However, to humans and most other multicellular species, biological aging remains an unstoppable process.

Whereas the process of biological aging is not a disease or health condition in and of itself, the biological dysfunctions and/or changes its progress entails are the cause of a number of diseases or health conditions.

At the theoretical level, it has been proposed that biological aging is a byproduct of a time-dependent imbalance of higher-order constraints on chromatin dynamics within the nucleus of each cell in multicellular species. In turn, this imbalance has been proposed to be strongly dependent on the higher-order constraints imposed by the somatic, replication-independent variants of the histone H1 protein (i.e., the histone variants H1.0 and H1.x and their protein orthologs). Specifically, these histone H1 protein variants—slowly but uninterruptedly during adulthood—decrease their affinity to nucleosomal and linker DNA in chromatin by virtue of the net accumulation of post-translational modifications (PTMs), which decrease the electrostatic binding affinity of the histone H1 protein to the negatively charged DNA (at physiological pH).

Artificial protein sequences and artificial nucleic acid sequences for the linker histone variants H1.0 (also known as histone H1′; H1(0); H5; H1δ; RI H1; or H1 histone family, member 0) and H1x (also known as histone H1.10 or H1 histone family, member X) have been proposed. In particular, proposed artificial protein sequences feature engineered α-helical motifs—three structural motifs in the histone H1 that bind to nucleosomal and/or linker DNA in chromatin. These artificial-sequence histone H1 proteins, when they replace or supplement their wild-type counterparts in vivo, confer multicellular individuals significant resistance to biological aging. Such sequences can be delivered via CRISPR/Cas9 genome editing or mRNA delivery. However, there is a need for alternative approaches that prevent a decrease in the histone H1.0/H1x DNA-binding affinity as the result of the accumulation of PTMs having cost-effective delivery mechanisms.

### SUMMARY OF THE INVENTION

Certain embodiments disclosed herein are directed to the specific use of specific cyclin-dependent kinase inhibitors; in particular, the use of CDK9 inhibitors and/or CDK5 inhibitors.

The use of CDK9 inhibitors and CDK5 inhibitors (separately or in combination) as described herein is aimed to the slowdown or prevention of biological aging.

Accordingly, some embodiments described herein include a method of treating, preventing, resisting, or slowing the progression of senescence, and/or age-related health conditions, comprising administering to a subject in need thereof a CDK inhibitor. In some embodiments, the senescence comprises skin senescence. In some embodiments, the CDK inhibitor is a CDK9 inhibitor. In some embodiments, the CDK9 inhibitor is a selective CDK9 inhibitor. In some embodiments, the CDK inhibitor is a CDK5 inhibitor. In some embodiments, the CDK5 inhibitor is a selective CDK5 inhibitor. In some embodiments, the CDK inhibitor is a CDK pan-inhibitor. Some embodiments include administering the subject a selective CDK5 inhibitor in combination with a selective CDK9 inhibitor. In some embodiments, the CDK inhibitor is both a CDK5 and a CDK9 inhibitor. In some embodiments, the CDK inhibitor is a selective CDK5 and CDK9 inhibitor. In some embodiments, the dose of the CDK inhibitor is selected to achieve a maximum serum concentration that is less than an IC50 of the inhibitor.

Embodiments described herein can also be used in general research for better understanding of the biological process.

Embodiments described herein also encompasses any clinical and cosmetic uses of the administration of a composition containing CDK9 inhibitors and/or CDK5 inhibitors.

### DETAILED DESCRIPTION OF THE INVENTION

Some embodiments include the use of one or more compounds that are CDK9 inhibitors and/or CDK5 inhibitors for the slowdown or prevention of biological aging.

Since the histone H1.0 and histone H1x proteins—slowly but uninterruptedly during adulthood—decrease their affinity to nucleosomal and linker DNA in chromatin by virtue of the net accumulation of post-translational modifications (PTMs), a plausible way to prevent such an accumulation is to inhibit the activity of the very enzymes that catalyze said PTMs.

Out of all PTMs proteins can undergo, phosphorylation is by far the most common and it also entails a dramatic decrease in the electric charge (at physiological pH) of the phosphorylated amino acid residue—which can be either Ser, Thr, Tyr, or His-- with respect to its post-translationally unmodified form. In this context, the post-translational phosphorylation of DNA-proximal amino acid residues in the histone H1.0/H1x thus decrease the electrostatic DNA binding affinity of the histone H1.0/H1x—a time-related phenomenon the compounds disclosed herein aims to prevent. The phosphorylating enzymes are generically called kinases, and if kinases are the target of inhibition, then the targeted kinases must be very few and highly specific, given the ubiquitousness of phosphorylation in vital biological processes, which in turn must not be significantly altered.

Both histone H1.0 and histone H1x proteins accumulate in terminally differentiated cells, i.e., their abundance in cells is replication-independent. Importantly, there are families of kinases known as cyclin-dependent kinases (CDKs), which are (i) constituted in their active form by a specific cyclin bound to the kinase itself, either strongly correlated or totally uncorrelated with the phases of the cell-cycle in terms of enzyme activity and, (iii) able to phosphorylate amino acid residues in the histone Hl. Since the histone H1.0 and histone H1x proteins accumulate in terminally differentiated cells, suitable targets for enzyme inhibition are those CDKs whose activity does not depend on the cell cycle, which are CDK5, CDK7, CDK8, and CDK9.

Among the CDK5, CDK7, CDK8, and CDK9 enzymes, the associated gene expression of two of them significantly correlates with that of the histone H1.0 and histone H1x proteins. Specifically, CDK9 significantly correlates in terms of associated gene expression with that of the H1.0/H1x histones across all tissues and CDK5 significantly correlates in terms of associated gene expression with that of the H1.0/H1x histones specifically across the central nervous system. Therefore, in some embodiments, CDK9 and CDK5 are the targets for enzyme inhibition.

Biological aging in humans is a relatively slow process: the changes it entails are close to negligible on a day-to-day basis. On the other hand, CDK9 and CDK5 have a number of phosphorylation targets other than the histone H1.0 and histone H1x proteins. From these two considerations, the use of CDK9 inhibitors and/or CDK5 inhibitors are used for the slowdown or prevention of biological aging. In some embodiments, the administration of the inhibitors is (i) very frequent (e.g., daily) and (ii) in very low doses (e.g., doses such that the concentration of the CDK9 inhibitor and/or the CDK5 inhibitor in vivo is significantly lower than the respective half-maximal inhibitory concentration, which is denoted by IC50). In various embodiments, the dose of the CDK inhibitor is less than 80%, 60%, 50%, 40%, 30%, 20%, 10%, 5%, 2.5%, 2%, 1.5%, 1%, 0.05%, 0.01%, or 0.001% of the IC50.

Some embodiments include the use of a CDK9 inhibitor and pharmaceutically acceptable salts thereof for the slowdown or prevention of biological aging, wherein the CDK9 inhibitor is exemplified by, but not limited to, the compounds LDC000067 (CAS No. 1073485-20-7), CDK-IN-2 (CAS No. 1269815-17-9), SNS-032 (CAS No. 345627-80-7), AZD4573 (CAS No. 2057509-72-3), Atuveciclib (CAS No. 1414943-94-4), BAY1251152 (CAS No. 1610358-56-9), MC180295 (CAS No. 2237942-08-2), JSH-150(CAS No. 2247481-21-4), CDK9-IN-1 (CAS No. 1415559-43-1), CDK9-IN-7 (CAS No. 2369981-71-3), CDK9-IN-8 (CAS No. 2105956-51-0), CDK9-IN-9 (CAS No. 2246956-84-1), and CDK9-IN-10 (CAS No. 3542-63-0).

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li+, Na+, K+, Mg2+and Ca2+and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

In some embodiments, the CDK9 inhibitor is a specific inhibitor. In some such embodiments, the CDK9 inhibitor specifically inhibits CDK9 to a greater extent than any other CDK protein by at least 1.2-fold, by at least 1.5-fold, by at least 1.8-fold, by at least 2-fold, by at least 3-fold, by at least 4-fold, by at least 5-fold, by at least 10-fold, by at least 15-fold, by at least 20-fold, by at least 50-fold, by at least 100-fold, by at least 150-fold, by at least 200-fold, by at least 400-fold, or by at least 500-fold.

In other embodiments, a CDK5 inhibitor and pharmaceutically acceptable salts thereof are used for the slowdown or prevention of biological aging, wherein the CDKS inhibitor is exemplified by, but not limited to, the compounds Roscovitine (CAS No. 186692-46-6) and Hymenidin (CAS No. 107019-95-4).

In some embodiments, the CDK5 inhibitor is a specific inhibitor. In some such embodiments, the CDK5 inhibitor specifically inhibits CDK5 to a greater extent than any other CDK protein by at least 1.2-fold, by at least 1.5-fold, by at least 1.8-fold, by at least 2-fold, by at least 3-fold, by at least 4-fold, by at least 5-fold, by at least 10-fold, by at least 15-fold, by at least 20-fold, by at least 50-fold, by at least 100-fold, by at least 150-fold, by at least 200-fold, by at least 400-fold, or by at least 500-fold.

In some embodiments, a CDK pan-inhibitor (i.e., a non-specific CDK inhibitor) that in particular inhibits the activity of CDK9 and/or inhibits that of CDK5, and pharmaceutically acceptable salts thereof, are used for the slowdown or prevention of biological aging, wherein the CDK pan-inhibitor is exemplified by, but not limited to, the compounds AT7519 (CAS No. 844442-38-2), Dinaciclib (CAS No. 779353-01-4), Flavopiridol (CAS No. 1431697-85-6), and CP668863 (also known as 20-223; CAS No. 865317-30-2).

In some embodiments, the CDK inhibitor inhibits both CDK9 and CDK5 to a greater extent that all other CDK proteins by at least 1.2-fold, by at least 1.5-fold, by at least 1.8-fold, by at least 2-fold, by at least 3-fold, by at least 4-fold, by at least 5-fold, by at least 10-fold, by at least 15-fold, by at least 20-fold, by at least 50-fold, by at least 100-fold, by at least 150-fold, by at least 200-fold, by at least 400-fold, or by at least 500-fold.

Some embodiments use a potent and highly-specific CDK9 inhibitor (e.g., LDC000067; CAS No. 1073485-20-7) or pharmaceutically acceptable salts thereof in the manufacture of compositions for the slowdown or prevention of biological aging. This embodiment aims to inhibit the CDK9 activity significantly and, above all, doing so as specifically as possible to avoid inhibiting additional CDK kinases other than CDK5. In general, if there is a trade-off between specificity and potency of a CDK inhibitor for its use as described herein, specificity should be preferred over potency. The choice of inhibiting only CDK9 in this embodiment relates to its whole-body significant enzyme activity (as opposed to CDK5 activity) and to the goal of not amplifying off-target inhibition—which in turn relates to the goal of preventing the phosphorylation of the histone H1.0 and histone H1x proteins, while neither the activity of CDK9 nor that of CDK5 is H1.0/H1x-specific.

Other embodiments use a combination of a highly-specific CDK9 inhibitor and a highly-specific CDK5 inhibitor for the slowdown or prevention of biological aging. In some embodiments, the highly-specific CDK9 inhibitor is LDC000067 (CAS No. 1073485-20-7) and the highly-specific CDK5 inhibitor is Roscovitine (CAS No. 186692-46-6) or respective pharmaceutically acceptable salts. This embodiment aims to provide additional protection to the central nervous system at the expense of augmenting the chances of off-target CDK inhibition.

Still other embodiments include the use of a CDK pan-inhibitor or pharmaceutically acceptable salts thereof that in particular inhibits the activity of CDK9 and CDK5 (e.g., Dinaciclib; CAS No. 779353-01-4) for the slowdown or prevention of biological aging. The use of CDK pan-inhibitors, whereas possibly less expensive, greatly amplifies the chances of off-target CDK inhibition and thus also the probability of adverse side effects, both in terms of diversity and severity.

### Administration and Pharmaceutical Compositions

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The selection of the appropriate dose of the compounds described herein is well within the knowledge of the skilled artisan. In some embodiments, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

### Methods of Treatment

Conditions that can be treated, prevented, resisted against, or protected against using the compounds described herein include senescence, and/or age-related health conditions. Accordingly, some embodiments include methods of treating, preventing, resisting, or slowing the progression of senescence, and/or age-related health conditions with the compounds and compositions comprising compounds described herein. Some embodiments include a method of treating, preventing, resisting, or slowing the progression of skin senescence with the compounds and compositions comprising compounds described herein. Some embodiments include a method of treating, preventing, resisting, or slowing the progression of skin wrinkles with the compounds and compositions comprising compounds described herein. Some embodiments include a method of increasing skin elasticity with the compounds and compositions comprising compounds described herein. Some embodiments include a method of reversing skin aging with the compounds and compositions comprising compounds described herein. Some methods include administering a compound or pharmaceutical composition described herein to a subject in need thereof. In some embodiments for use against skin senescence, the compounds and compositions described herein are administered topically to the skin. In some embodiments, a subject can be an animal, e.g., a mammal, e.g., a human. In some embodiments, the human is an adult. In some embodiments, the adult is older than 21 years, 22 years, 23 years, 24 years, 25 years, 26 years, 27 years, 28 years, 29 years, 30 years old, 31 years old, 32 years old, 33 years old, 34 years old, or 35 years old.

In this patent application, reference is made to the following naturally occurring α-amino acids (aa): histidine (IUPAC one-letter symbol: H, three-letter symbol: His), serine (S, Ser), threonine (T, Thr), and tyrosine (Y, Tyr).

Reference is made to a CAS Registry Number or simply CAS No., understood as a unique numerical identifier assigned by the Chemical Abstracts Service (CAS) to every chemical substance described in the open scientific literature.

Reference is made to the net electric charge defined as the algebraic sum of the charges present at the surface of a molecule divided by the elementary charge of the proton.

Reference is made to nucleosomal DNA (also known as core nucleosomal DNA), understood as the DNA that is left-hand wrapped around the histone octamer forming a complex known as the nucleosome core particle (NCP), which is the building block of chromatin.

Reference is made to linker DNA, understood as the DNA that extends in between nucleosome core particles. Importantly for this invention, the phosphate group repeated across the backbone of nucleic acids in particular makes both nucleosomal and linker DNA negatively charged at physiological pH.

Reference is made to the histone H1 (also known as linker histone) protein, which constitutes one of the five major histone protein families necessary for the formation of chromatin in the eukaryotic cell. Specific regions within the histone H1 protein bind to nucleosomal and/or linker DNA, which in turn stabilizes the higher-order constraints on chromatin dynamics.

The histone H1 family comprises a number of variants. In particular, reference is made to the variants H1.0 and H1x, which are the only variants whose associated gene expression is both somatic and replication-independent.

Reference is also made to post-translational modifications (PTMs), which are covalent and typically (but not necessarily) enzymatic modifications undergone by amino acid residues in proteins following protein biosynthesis.

### EXAMPLES

Example 1: In vivo testing of the use of a CDK9 inhibitor for conferring Caenorhabditis elegans (strain N2) individuals resistance to biological aging.

A survival assay (C. elegans individuals kept at 20° C. and fed with E. coli OP50) is conducted to assess whether there is a significant correlation between median C. elegans lifespan and the concentration of the CDK9 inhibitor LDC000067 (CAS No. 1073485-20-7; IC₅₀=44 [nM]) in the medium. Dimethyl sulfoxide (DMSO) is used as a solvent for LDC000067 in order to prepare the treated medium.

Example 2: In vivo testing of the use of a CDK5 inhibitor for conferring Caenorhabditis elegans (strain N2) individuals resistance to biological aging.

A survival assay (C. elegans individuals kept at 20° C. and fed with E. coli OP50) is conducted to assess whether there is a significant correlation between median C. elegans lifespan and the concentration of the CDK5 inhibitor Roscovitine (CAS No. 186692-46-6; IC₅₀<0.2 [μM]). Dimethyl sulfoxide (DMSO) is used as a solvent for Roscovitine in order to prepare the treated medium.

Example 3: In vivo testing of the use of a CDK pan-inhibitor for conferring Caenorhabditis elegans (strain N2) individuals resistance to biological aging.

A survival assay (C. elegans individuals kept at 20° C. and fed with E. coli OP50) is conducted to assess whether there is a significant correlation between median C. elegans lifespan and the concentration of the CDK pan-inhibitor AT7519 (CAS No. 844442-38-2; CDK9 IC₅₀=10 [nM]; CDK5 IC₅₀=13 [nM]). Dimethyl sulfoxide (DMSO) is used as a solvent for AT7519 in order to prepare the treated medium.

What is claimed is:

1. A method of treating, preventing, resisting, or slowing the progression of senescence, and/or age-related health conditions, comprising administering to a subject in need thereof a CDK inhibitor.

2. The method of claim 1, wherein the senescence comprises skin senescence.

3. The method of claim 1 or 2, wherein the CDK inhibitor is a CDK9 inhibitor.

4. The method of claim 3, wherein the CDK9 inhibitor is a selective CDK9 inhibitor.

5. The method of claim 3, wherein the CDK9 inhibitor is selected from the group consisting of LDC000067, A-1592668, A-1467729, Wogonin, CDKI-73, LY2857785, CDK-IN-2, SNS-032, AZD4573, Atuveciclib, BAY1251152, MC180295, JSH-150, CDK9-IN-1, CDK9-IN-6, CDK9-IN-7, CDK9-IN-8, CDK9-IN-9, CDK9-IN-10, CAY10574, PHA-767491, and pharmaceutically acceptable salts thereof.

6. The method of claim 3, wherein the CDK9 inhibitor is LDC000067 of a pharmaceutically acceptable salt thereof.

7. The method of claim 1 or 2, wherein the CDK inhibitor is a CDK5 inhibitor.

8. The method of claim 7, wherein the CDK5 inhibitor is a selective CDK5 inhibitor.

9. The method of claim 7, wherein the CDK5 inhibitor is selected from the group consisting of Roscovitine, Hymenidin, Indirubin-3′-monoxime, PHA-793887, and pharmaceutically acceptable salts thereof.

10. The method of claim 1 or 2, wherein the CDK inhibitor is a CDK pan-inhibitor.

11. The method of claim 10, wherein the CDK pan-inhibitor is selected from the group consisting of AT7519, Dinaciclib, Flavopiridol, CP668863, and pharmaceutically acceptable salts thereof.

12. The method of claim 1 or 2, comprising administering the subject a selective CDK5 inhibitor in combination with a selective CDK9 inhibitor.

13. The method of claim 12, comprising administering the subject LDC000067 or a pharmaceutically acceptable salt thereof and Roscovitine or a pharmaceutically acceptable salt thereof.

14. The method of claim 1 or 2, wherein the CDK inhibitor is both a CDK5 and a CDK9 inhibitor.

15. The method of claim 14, wherein the CDK inhibitor is a selective CDK5 and CDK9 inhibitor.

16. The method of claim 1 or 2, comprising administering a dose of the CDK inhibitor to achieve a maximum serum concentration that is less than an IC₅₀ of the inhibitor.

17. Use of a CDK inhibitor in the preparation of a medicament for treating, preventing, resisting, or slowing the progression of senescence, and/or age-related health conditions.

18. A CDK inhibitor for use in treating, preventing, resisting, or slowing the progression of senescence, and/or age-related health conditions.
